(19) United States
(12) Patent Application Publication
(10) Pub. No.: US 2024/0363233 A1
(43) Pub. Date: Oct. 31, 2024

(54) Digital Patient Pathway in personalized healthcare systems with Advanced Medical Manufacturing Simulator

(71) Applicant: THINKING ROBOTSTUDIOS INC. AND MASH.FLIGHTS, Truro (CA)

(72) Inventors: Kendall Joudrie, Nova Scotia (CA); Jourdan Joudrie, Halifax (CA)

(73) Assignee: THINKING ROBOTSTUDIOS INC. AND MASH.FLIGHTS, Truro, NS (CA)

(21) Appl. No.: 18/647,896

(22) Filed: Apr. 26, 2024

(30) Foreign Application Priority Data
Apr. 28, 2023 (US) 63462877

(51) Int. Cl. G16H 40/20; G16H 10/60; G16H 50/30

(52) U.S. Cl. CPC G16H 40/20; G16H 10/60; G16H 50/30

(57) ABSTRACT

A system and method for generating customized treatments for patients by integrating two AI-driven data systems, herein referred to as Digital Twins. The first Digital Twin encompasses a comprehensive reflection of the patient's health condition, incorporating medical data, genetic information, imaging results, health history, lifestyle factors, and patient-reported complaints. Utilizing this Digital Twin artificial intelligence algorithms, analyzes cross-references between medical conditions and potential treatments to generate digital personalized treatment pathways. Furthermore, it predicts and recommends preventive measures and lifestyle adjustments. The second Digital Twin represents the capabilities of a pharmaceutical manufacturing company or a healthcare enterprise, possessing expertise in producing pharmaceuticals and medical devices. Through AI-driven processes, this Enterprise Digital Twin organizes the production of treatments proposed by the Digital Patient Pathway, ensuring customization and efficiency. This includes but is not limited to manufacturing patient-specific medications, orthopedic implants, or surgical plans.

### FIELD OF INVENTION

This invention relates to a digital patient healthcare system with medical manufacturing simulation; and in particular digital twins in healthcare for patient specific digital twins and an organizational digital twin. This invention also relates to connecting two AI driven digital twins into producing an effective treatment for patients.

### BACKGROUND

Generally speaking a digital twin is a digital model of an intended or actual real-world physical product, system, or process that serves as the effectively indistinguishable digital counterpart of it for practical purposes, such as simulation, integration, testing, monitoring, and maintenance.

The healthcare industry is facing unprecedented challenges in providing personalized care and effective treatment for patients. Digital twins have emerged as a promising solution to address these challenges by creating virtual models of patients, medical devices, and healthcare systems based on real-world data.

### SUMMARY

With the integration of artificial intelligence and the Internet of Things, digital twins may provide real-time insights and decision-making support for healthcare professionals, as well as optimize organizational strategies and predict future outcomes.

This disclosure presents an approach for generating AI-generated digital twins in healthcare in combination with direct medical device and medication production. A method leverages data from a variety of sources, including medical imaging scans and health records, to create personalized digital twins that can guide treatment planning and provide predictive analytics. The disclosure describes an approach which can be extended to optimize the production and supply of medical devices and drugs, improving patient outcomes while reducing costs and increasing safety. This is only made possible by integrating comprehensive broad field diagnostic, treatment capabilities, and advanced manufacturing, all of which are data harvested on a per patient level. The consistent and proactive comprehensive data equates to a stable virtual healthcare simulation, eliminating errors from outside sources, and filling diagnostic gaps from lack of testing. Traditional healthcare has become reactive, diagnostic, and treatment for the presented issue not proactive whereas comprehensive diagnostic is harvested from the same tests to fill in the patient data and prediction model, which in turn allows for full customized treatment for existing and future health prediction.

It is an aspect of this invention to provide digital twins in healthcare for a patient comprising: a patient-specific digital twin and an organizational digital twin.

It is another aspect of this invention to A method for generating customized treatments for patients by integrating two AI-driven data systems, comprising; to create an algorithm based on this description, we can break down the process into discrete steps; collecting various types of patient data including medical tests, genetic information, imaging results, health records, family medical history, lifestyle factors, and patient-reported symptoms; analyzing the collected data to create a comprehensive representation of the patient's health condition; Identifying correlations between the patient's medical conditions and potential treatment options; predicting future health risks and recommend treatment preventive measures or lifestyle modifications tailored to the individual patient.

### BRIEF DESCRIPTION OF DRAWINGS

Embodiments of the disclosure will now be described, by way of example only, with reference to the accompanying drawings, in which:

FIG. 1 is a schematic chart of a digital patient pathway diagram for patient data according to an embodiment;

FIG. 2 is a schematic chart of a digital patient pathway input and output algorithm according to an embodiment;

FIG. 3 is a schematic chart of a Healthcare enterprise digital twin input and output algorithm according to an embodiment; and

FIG. 4 is a schematic chart of a healthcare enterprise manufacturing and supply chain organizational diagram according to an embodiment.

### OVERVIEW

This disclosure is related to use of two main digital twins in healthcare: a patient-specific digital twin and an organizational digital twin. The patient's digital twin is generated through AI and define the digital patient pathway, system or method based on data gathered from advanced healthcare systems through comprehensive diagnostic and treatment capability to harvest said data, including the patient's medical history which includes, but not limited to items, data, and information described herein.

Once defined the digital patient pathway is then used to drive the input for an organizational digital twin, which is generated also through AI and uses available fabrication resources to simulate, analyze and produce optimized solutions for the patient's case, including medication, surgery planning, instrumentation for the surgery, and prescriptions and predictions for further treatments and healing provided by said advanced healthcare systems. This approach allows diagnosis and therapy selection, procedure planning and guidance to be tailored to the individual patient's needs and preferences and materialize suggested personalized treatment plans and devices, hence, improved patient outcomes, reducing costs, and increased safety.

Digital twins have become increasingly popular in various industries, as they offer an accurate and efficient way to represent real-world entities and activities. In healthcare, digital twins, or the digital patient pathway can be used to model patients, medical devices, and healthcare systems based on data gathered from the real world. This technology has a vast potential to improve patient care and research, optimize organizational strategies, and predict future outcomes.

The integration of the internet of things (IoT), artificial intelligence (AI), and complex data is crucial for creating insights and supporting real-time decision-making in the healthcare industry. The digital patient pathway approach can help to achieve personalized diagnosis and therapy selection to procedure planning and guidance tailored to the patient's physical characteristics, medical history, current condition, and future needs. Specifically designed, produced, and delivered medical devices and medications can then be effectively applied to meet the patient's preferences and needs.

An advanced healthcare organization allows for comprehensive and stable data collection as it controls all data collection, diagnostics, and properly addresses it to advanced manufacturing integration and output.

Digital patient pathway is created by collecting data from MRI scans, CT scans and other medical imaging devices, medical examination, and tests such as but not limited to the detailed below:
Scan data, including merged data from CT, X-ray, MRI, Ultrasound, PET scans, others
Observation and physical examination
Gait analysis
Height, weight, age, Body Mass Index (BMI)
Material toxicity test
Use of medications
Comorbidities and prior disease states, previous surgeries, bone density and bone lose data
Ethnicity and DNA
Medical history
Activity history and goals.
Physical characteristics, and current condition.
Blood tests (CBC, liver function tests, thyroid function tests, cholesterol screening, etc.)
Urine tests (urinalysis, drug tests)
Electrocardiogram (ECG or EKG)
Electroencephalogram (EEG)
Biopsy
Colonoscopy
Pap smear
Mammogram
Pulmonary function tests
Genetic testing
Endoscopy

Further analysis from the AI including symptomatic data, statistics, and the health record of the patient can be formulated to develop a research question and hypothesis, on which diagnosis is made. Based on that, optimal computed results are proposed, and guidance for treatment is created. The latter is also an input for further improvement of the digital patient pathway by means of machine learning algorithms. In addition, through plurality simulations and optimizations, the digital patient pathway by AI predicts future medical conditions and determines preventive actions and makes future treatment suggestions. This approach can help medical professionals to review, approve, or edit the treatment plans to refine and provide alternative approaches if needed and possible.

Moreover, AI can simulate hypothetical processes to observe the behavior with a given treatment, calculate the expected outcomes or compare them with the existing processes. For example, this technology can be used to simulate implants for fit and integrity, and materials to be used in manufacturing them, to avoid infection and toxicity.

The organizational digital twin integrates data in real-time from various digital sources in the healthcare enterprise organization, such as but not limited to enterprise resource planning and supply chain management systems. Advanced analytics and machine learning algorithms process and analyze that data to simulate and optimize further production or supply of but not limited to medical devices, drugs based on the treatment plans for each patient or for a group of patients when commonality is detected. The organizational digital twin is used to simulate based on the available manufacturing processes and make suggestions for manufacturing planning, requisitions for purchasing, and generate shop floor documentation.

In conclusion, the implementation of digital twins in healthcare provides significant benefits for both patients and healthcare providers. By using this technology, healthcare professionals make informed decisions based on real-time data, which leads to improved patient outcomes, reduced healthcare costs, and increased productivity.

### DETAILED DESCRIPTION OF THE INVENTION

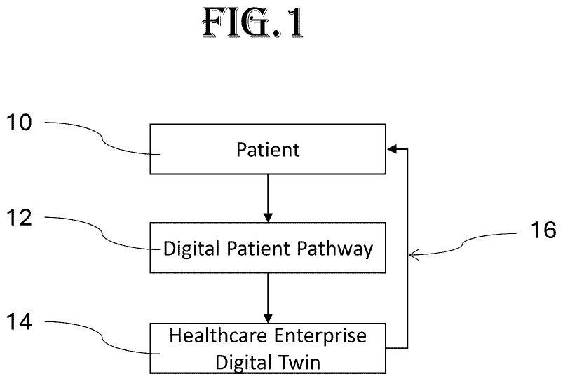

FIG. 1 schematically presents a system comprising a patient 10, a digital patient pathway 12, and the generation of a healthcare enterprise digital twin (14). Patient treatment is represented by the arrow 16.

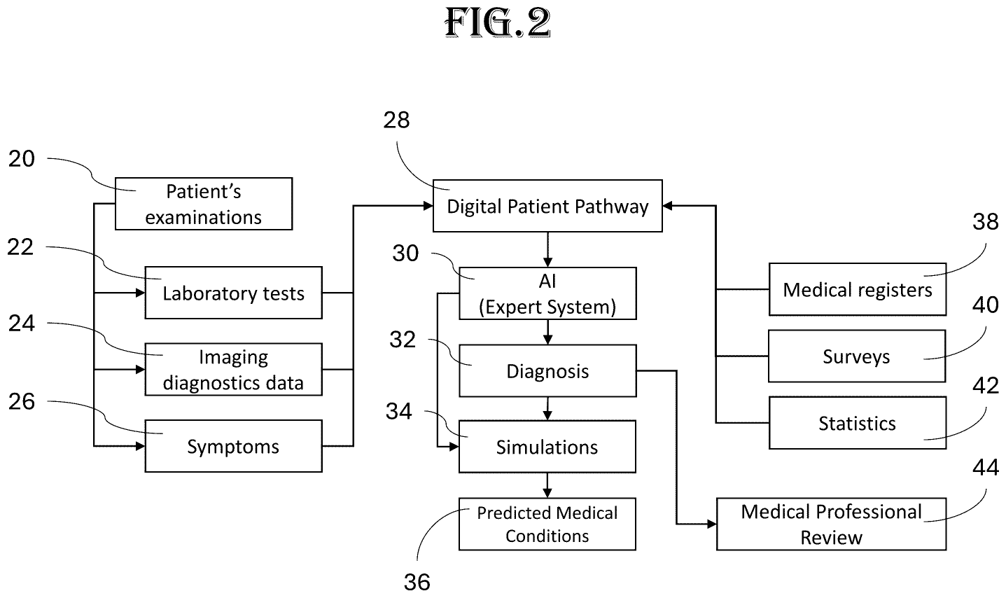

FIG. 2 details the flow of data from patient examinations (20), laboratory tests (22), imaging diagnostics (24), and symptoms (26) into a digital patient pathway (28). This pathway incorporates an AI expert system (30) to provide diagnosis (32), simulations (34), and predicted medical conditions (36), culminating in a medical professional review (44). Additional inputs such as medical records (38), surveys (40), and statistical data (42) continually enhance the Digital Patient Pathway (28).

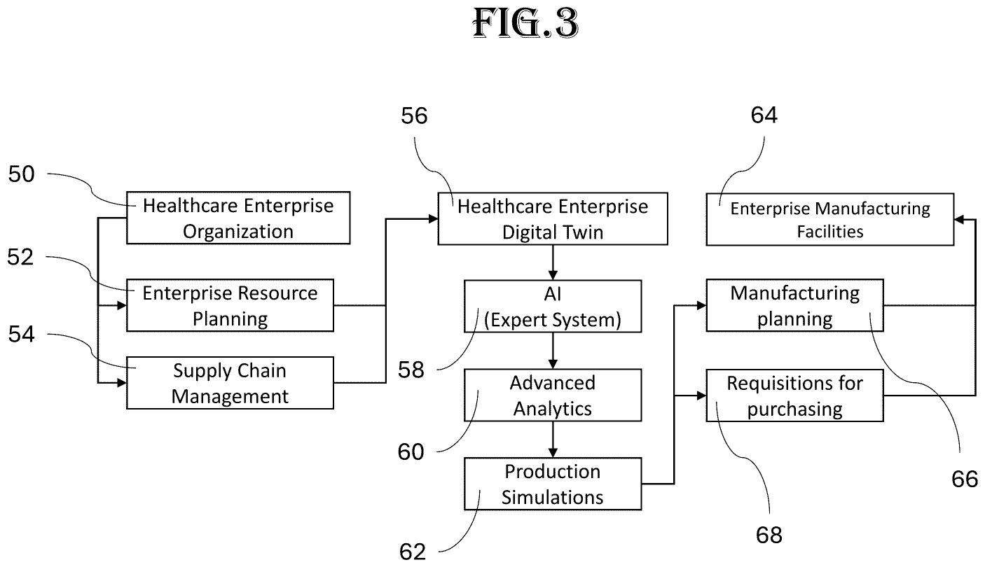

FIG. 3 illustrates the Healthcare Enterprise Organization 50, which encompasses Enterprise Resource Planning 52 and Supply Chain Management 54. Utilizing the Healthcare Enterprise Digital Twin 56, which integrates an AI expert system 58 and Advanced Analytics 60, the enterprise generates production simulations 62. These simulations inform manufacturing planning 66 and requisitions for purchasing 68 at the enterprise manufacturing facilities 64.

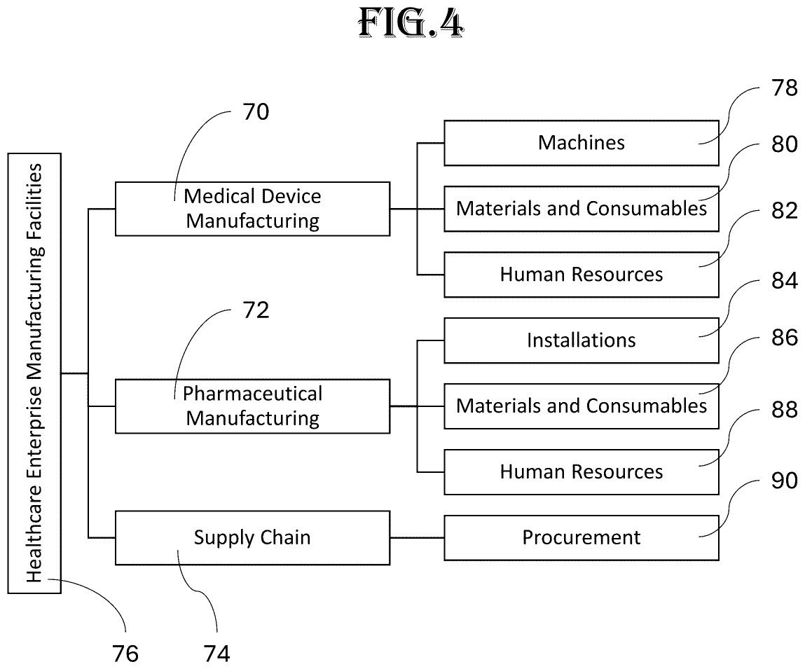

FIG. 4 represents the Healthcare Enterprise Manufacturing Facilities 76 linked to medical device manufacturing 70, pharmaceutical manufacturing 72, and supply chain operations 74. The medical device section 70 includes machines 78, materials and consumables 80, and human resources 82. The pharmaceutical section 72 features installations 84, materials and consumables 86, and human resources 88. The supply chain 74 involves procurement 90.

As stated above the digital patient system or pathway in personalized healthcare systems with advanced medical manufacturing is about connecting two AI driven digital twins into producing effective treatment for patients.

The first digital twin reflects the patient and more specifically their health condition. The digital twin is construct of data collected from various medical tests, DNA tests, imaging data (including X-ray/CT/MRI/PET scans, but already analyzed and data is segmented), patient's health history, previous conditions, family health history, lifestyle, etc., and the most important—the patient's complaints. From specifically developed expert systems or AI we want extensive searches for all the cross references between the medical conditions of the respective digital twin with possible treatments, analyses of the results, diagnosis, and simulation. This process forms the core of what is termed the “Digital Patient Pathway”. In addition to its diagnostic and treatment recommendation functions, this digital twin also leverages predictive analytics to propose preventive measures and suggest lifestyle modifications tailored to the individual's health profile, enhancing proactive healthcare management.

The second digital twin embodies the operational and production capabilities of a sophisticated pharmaceutical and medical device manufacturing enterprise, herein referred to as a Healthcare Enterprise. This digital twin enables the enterprise to efficiently produce a diverse range of medical products, from pharmaceuticals to orthopedic implants, tailored to the specific needs of patients. Utilizing advanced artificial intelligence (AI), this digital twin aggregates and analyzes all relevant manufacturing process knowledge. The AI is tasked with organizing and overseeing the production of customized medical treatments that have been specified by the patient's digital twin within the Digital Patient Pathway. This includes the formulation of specific medications in precise dosages, the creation of patient-specific implants, or the preparation of detailed surgical plans employing a “surgery-in-a-box” approach. In scenarios where the proposed standard treatments are insufficient or impractical, the AI within this digital twin is also responsible for generating alternative solutions. This ensures that the healthcare enterprise can adapt to various patient needs and medical challenges. Furthermore, while the AI plays a critical role in optimizing and fine-tuning treatment plans and production processes. A hybrid approach allows for the precision and speed of AI to be combined with the nuanced judgment of human experts. The system is designed to maximize efficiency and accuracy in treatment production, leveraging controlled data to make adjustments that are beyond the typical capabilities of human operators, thereby reducing the potential for human error and technical discrepancies.

In one embodiment utilizing the “closed loop” system, the configuration includes state-of-the-art equipment and advanced AI detection systems designed to enhance the accuracy and effectiveness of medical diagnostics. This system mandates the performance of specific diagnostic tests that provide extensive data coverage—far exceeding standard practices. For instance, a single blood work analysis in this system can evaluate over 300 biomarkers, compared to typically fewer than ten in a standard lab visit. Additional tests include DNA profiling that offers insights into ethnic clinical data markers, material toxicology to ensure compatibility of medical materials with patient biology, as well as advanced assessments of bone and muscle density and comprehensive imaging technologies.

This meticulous approach to data collection and analysis standardizes the information gathered, thereby facilitating the systematic development and refinement of the digital patient pathway. The AI systems integrated within the second digital twin utilize this standardized data to generate highly precise suggestions for medical treatments. These suggestions are devoid of discrepancies that often arise from varied data sources, enabling the precise manufacturing of customized treatments such as pharmaceuticals, implants, stem cell therapies, and bespoke vaccines.

Furthermore, this closed loop system allows for the dynamic updating and refining of the digital twin models. As the patient undergoes treatment and further diagnostics, the collected data feeds back into the system, allowing continuous adjustment and optimization of the treatment plans based on real-time patient responses and outcomes. This ongoing cycle enhances the precision of clinical diagnostics and treatment, ensuring that each patient's digital twin evolves to more accurately reflect their individual health status and needs, thereby achieving optimal therapeutic efficacy.

The invention described herein describes treatment customization by integrating two distinct AI-driven expert systems. The first Digital Twin serves as a comprehensive reflection of the patient's health status, drawing upon a multitude of data sources including medical tests, genetic analyses, imaging data (such as X-ray, CT, MRI, PET scans), health records, family medical history, lifestyle factors, and patient-reported symptoms. Employing state-of-the-art artificial intelligence techniques, this Digital Twin conducts extensive searches to identify correlations between medical conditions and potential treatment options. This process, referred to as the Digital Pathway, mimics the diagnostic and treatment decision-making capabilities akin to those of a highly skilled medical professional, akin to the fictional character Dr. House. Moreover, the first Digital Twin employs predictive modeling to anticipate future health risks and recommends preventive measures or lifestyle modifications tailored to the individual patient.

The second Digital Twin encapsulates the expertise and capabilities of a pharmaceutical manufacturing company or a healthcare enterprise specializing in medical device production. This Digital Twin leverages advanced AI algorithms to streamline the production process, facilitating the creation of customized treatments identified by the patient's Digital Pathway. Whether it involves manufacturing bespoke medications, patient-specific orthopedic implants, or even surgical plans using innovative approaches like the “surgery-in-a-box” paradigm, this Digital Twin ensures the seamless translation of treatment plans into actionable solutions. In instances where the primary healthcare enterprise faces limitations in providing the proposed treatment, the second Digital Twin offers alternative options and solutions, thereby enhancing the overall efficacy and adaptability of the treatment generation process.

The integration of these two Digital Twins improves the landscape of personalized medicine, enabling healthcare providers to deliver tailored treatments with unprecedented precision and efficiency. By harnessing the synergies between advanced AI technologies and domain-specific expertise, the Digital Patient Pathway described herein represents a paradigm shift in healthcare delivery, poised to significantly enhance patient outcomes and improve the standard of care.

### System Method and Algorithm

The description provided for the Digital Patient Pathway patent outlines a system and method for generating customized treatments for patients by connecting two AI-driven expert systems, Digital Twins. To create an algorithm based on this description, we can break down the process into discrete steps:
1. Data Acquisition: Collect various types of patient data including medical tests, genetic information, imaging results, health records, family medical history, lifestyle factors, and patient-reported symptoms.
2. Digital Pathway Analysis:
   Analyze the collected data to create a comprehensive representation of the patient's health condition.
   Utilize advanced artificial intelligence algorithms to identify correlations between medical conditions and potential treatment options.
   Predict future health risks and recommend preventive measures or lifestyle modifications tailored to the individual patient.
3. Treatment Generation:
   Upon receiving the diagnosis and treatment recommendations from the Digital Pathway analysis, determine the feasibility and effectiveness of the proposed treatment options.
   If necessary, explore alternative treatment solutions based on the patient's specific health profile and constraints.
4. Production Organization:
   Utilize AI-driven processes to organize the production of treatments proposed by the Digital Pathway.
   Customize the production process to create patient-specific medications, orthopedic implants, surgical plans, or other tailored treatments.
5. Quality Assurance:
   Implement quality control measures to ensure the safety, efficacy, and compliance of the produced treatments with regulatory standards.
   Continuously monitor and optimize the production process based on feedback and performance metrics.

Put another way an algorithm to encapsulate the above invention includes the steps of: Input: Patient data (medical tests, genetic information, imaging results, health records, etc.)
1. Data Acquisition:
   Collect patient data
2. Digital Pathway Analysis:
   Analyze patient data to create a health profile
   Apply AI algorithms to identify correlations between medical conditions and treatments
   Predict future health risks and recommend preventive measures
3. Treatment Generation:
   Receive diagnosis and treatment recommendations from Digital Pathway
   Evaluate feasibility and effectiveness of proposed treatments
   Explore alternative treatment options if necessary
4. Production Organization:
   Organize production of treatments based on Digital Pathway recommendations
   Customize production process for patient-specific treatments
5. Quality Assurance:
   Implement quality control measures for produced treatments
   Monitor and optimize production process

Output: Customized treatment plan for the patient

This algorithm outlines the steps involved in the Digital Pathway patent, focusing on the technical processes and interactions between the AI-driven expert systems to generate tailored treatments for patients. It emphasizes the integration of advanced AI techniques with healthcare expertise to deliver personalized and effective medical interventions,

The invention is described in relation to the embodiments outlines above but should not be limited to those embodiments but rather by the invention as claimed.

1. Digital twins in healthcare for a patient comprising: a patient-specific digital twin and an organizational digital twin.

2. The digital twins as claimed in claim 1 wherein said digital twins are patient digital twins generated through AI and define the digital patient pathway based on data gathered from advanced healthcare systems through comprehensive diagnostic and treatment capability to harvest said data, including the patient's medical history.

3. The digital twins as claimed in claim 2 where once said digital twins are defined the digital patient pathway is then used to drive the input for an organizational digital twin, which is generated also through AI and uses available fabrication resources to simulate, analyze and produce optimized solutions for the patient's case, including medication, surgery planning, instrumentation for the surgery, and prescriptions and predictions for further treatments and healing provided by said advanced healthcare systems, wherein the digital twins enable diagnosis and therapy selection, procedure planning and guidance to be tailored to the patient's needs and preferences and materialize suggested personalized treatment plans and devices, providing improved patient outcomes, costs reductions, and increased safety.

4. A method for generating customized treatments for patients by integrating two AI-driven data systems, comprising; to create an algorithm based on this description, we can break down the process into discrete steps:
a) collecting various types of patient data including medical tests, genetic information, imaging results, health records, family medical history, lifestyle factors, and patient-reported symptoms;
b) analyzing the collected data to create a comprehensive representation of the patient's health condition.
c) Identifying correlations between the patient's medical conditions and potential treatment options.
d) predicting future health risks and recommend treatment preventive measures or lifestyle modifications tailored to the individual patient.

5. The method as claimed in claim 4 upon receiving the diagnosis and treatment recommendations, determine the feasibility and effectiveness of the proposed treatment options.

6. The method as claimed in claim 5 comprising:
a) optionally explore alternative treatment solutions based on the patient's specific health profile and constraints.

7. The method as claimed in claim 6 comprising utilize AI-driven processes to organize the production of treatments proposed.

8. The method as claimed in claim 7 comprising creating patient-specific medications, orthopedic implants, surgical plans, or other tailored treatments.

9. The method as claimed in claim 8 comprising implementing quality control measures to ensure the safety, efficacy, and compliance of the treatments with regulatory standards.

10. The method as claimed in claim 9 continuously monitoring and optimizing by feedback and selected performance metrics.

11. A system comprising hardware and software connected to the internet to operate the method described.
